(19) United States
(12) Patent Application Publication
(10) Pub. No.: US 2015/0073399 A1
(43) Pub. Date: Mar. 12, 2015

(54) Hand-Held Portable Laser Surgical Device

(71) Applicant: Den-Mat Holdings, LLC, Lompoc, CA (US)

(72) Inventors: Mihai I. A. Boitor, Martinez, CA (US); Alexandre B. Di Sessa, Walnut Creek, CA (US)

(73) Assignee: DEN-MAT HOLDINGS, LLC, Lompoc, CA (US)

(21) Appl. No.: 14/547,421

(22) Filed: Nov. 19, 2014

Related U.S. Application Data
(63) Continuation of application No. 12/258,111, filed on Oct. 24, 2008.

(51) Int. Cl. A61B 18/22

(52) U.S. Cl. CPC A61B 18/22; A61B 2017/0046
USPC 606/15

(57) ABSTRACT

A hand-held, portable diode laser surgical device includes a power supply, at least one laser diode, integral control interface and display, and a multi-component sterile, disposable tip apparatus featuring assembly for alignment of a self-contained optical fiber to the surgical device, and releasably locking assembly between the tip apparatus and surgical device. An embodiment includes wireless foot pedal on/off control and a dock providing sterile, antiseptic recharging environment.

### CROSS-REFERENCES TO RELATED APPLICATIONS

None.

### STATEMENT REGARDING FEDERALLY SPONSORED RESEARCH OR DEVELOPMENT

None.

### REFERENCE TO A MICRO-FICHE APPENDIX

None.

### BACKGROUND OF THE INVENTION

1. Field of the Invention

This invention relates to portable laser surgical instruments. More particularly, the invention relates to an improved, hand-held laser surgical device combining at least one diode laser, power supply, and replaceable, disposable sterile tip with protective sleeve contained within a pen sized unit, particularly adaptable to oral surgery.

2. Description of the Related Art Including Information Disclosed Under 37 C.F.R. 1.97 and 1.98

A search of the prior art located the following United States patents which are believed to be representative of the present state of the prior art: U.S. Pat. No. 6,325,791 B1, issued Dec. 4, 2001; U.S. Pat. No. 6,458,120 B1, issued Oct. 1, 2002; U.S. Pat. No. 6,213,998 B1, issued Apr. 10, 2001; U.S. Pat. No. 7,118,563, issued Oct. 10, 2006; U.S. Pat. No. 5,616,141, issued Apr. 1, 1997; U.S. Pat. No. 5,607,420, issued Mar. 4, 1997; U.S. Pat. No. 6,013,096, issued Jan. 11, 2000; U.S. Pat. No. 5,464,436, issued Nov. 7, 1995; U.S. Pat. No. 5,304,172, issued Apr. 19, 1994; U.S. Pat. No. 7,033,350 B2, issued Apr. 25, 2006; U.S. Pat. No. 6,572,637 B1, issued Jun. 3, 2003; U.S. Pat. No. 6,574,401 B1, issued Jun. 3, 2003; U.S. Pat. No. 6,868,221 B1, issued Mar. 15, 2005; U.S. Pat. No. 6,261,310 B1, issued Jul. 17, 2001; U.S. Pat. No. 7,267,672 B2, issued Sep. 11, 2007; U.S. Patent Publication 2004/0259053 A1, published Dec. 23, 2004; U.S. Pat. No. 5,927,977, issued Jul. 27, 1999; U.S. Pat. No. 6,746,473 B2, issued Jun. 8, 2004; and International Patent Publication No. WO 01/10327 A1, published Feb. 15, 2001.

### BRIEF SUMMARY OF THE INVENTION

Medical laser treatment using hand-held instruments has generally been developed for ophthalmic, dental, orthopedic, and similar surgical procedures where the treatment area is confined or particularly difficult to reach. Typically, a laser beam is transmitted from a laser source though an optical fiber to a treatment site. The optical fiber terminates proximally in a laser source connector for connection to the laser source and terminates distally in a hand-piece manipulated by the surgeon.

The diode laser has been in use for medical and dental purposes. Specifically, in the area of dental use, the diode lasers on the market have been reduced in size to approximately that of a shoe box. The dental laser unit has typically been connected to the delivery or surgical device in the form of a hand-piece using an optical fiber.

Laser surgical devices provide certain advantages over traditional implements such as irradiation to vaporize the tissue and small blood vessels proximate thereto. Accordingly, laser surgical devices provide operations performed without hemorrhaging. Thus, the underlying conditions requiring blood transfusions to patients vanishes. Bloodless surgery also has the effect of preventing disease, which can be disseminated by transfusions or infections at the surgical site. These advantages are particularly useful for oral and dental surgical procedures, such as troughing, implant exposure, restoration, or pyogenic granuloma.

Another important advantage of medical laser devices is that small lymphatic vessels in tissues are sealed by the irradiation of the laser beams. This feature greatly reduces occurrence of edema caused by accumulation of lymph after surgery, and likewise prevents spreading of cancerous cells during surgery or thereafter.

Laser surgery often reduces post-surgical pain by sealing nerve ends cut at the operational target site.

Post-operative scar tissue is reduced by use of medical laser devices. Accordingly, the incidence of post-surgical stricture is reduced, thus minimizing a major cause of re-surgery.

Often, a laser surgical hand-piece used during one procedure cannot be used with another patient in a subsequent procedure unless some form of sterilization is performed. Types of sterilization techniques range from autoclaves to gas. Gas procedures are time consuming and costly. Autoclave temperatures generally have proven too severe for laser surgical hand-pieces to withstand.

Accordingly it would be useful to provide a hand-held laser surgical device, particularly suitable for cutting soft tissue, that eliminates the need for a base laser unit.

It would be of further benefit if the hand-held laser surgical device had a separate wireless foot pedal to activate the on-off switch for the surgical device, a small screen and several control buttons for surgeon interface with the instrument, thus providing a dental soft-tissue laser surgical device that is simpler and easier to use.

Another desired aspect for a hand-held laser surgical device would be a sterile, disposable tip for use with the handheld laser surgical device.

It would be of further use if the sterile, disposable tip provided means for precise alignment of the optical fiber in the tip to the source of laser energy in the surgical device.

Yet another useful advantage would be for the sterile, disposable tip to be releasably attached to the device with mechanical, magnetic, electromechanical, or electromagnetic locking assembly.

Finally, when not in use, the hand-held laser surgical device is stored in a rechargeable power supply base which offers ultra violet (UV) light for additional cleanliness and sterilization.

Accordingly it would be useful to provide a hand-held laser surgical device for use with a sterile, disposable tip having a quick and easy to use alignment and connection assembly for use the laser surgical device, such as our disposable surgical tip apparatus disclosed and claimed in pending U.S. Non-provisional patent application Ser. No. 12/115,336, filed on May 5, 2008 [“the '336 Application”] which is incorporated herein by reference for all purposes.

Further, it would be useful to provide a hand-held laser surgical device for use with a dual diode converging module, such as our dual diode converging module disclosed and claimed in pending U.S. Non-provisional patent application Ser. No. 12/115,383, filed on May 5, 2008 [“the '383 Application”] which is incorporated herein by reference for all purposes.

It would be yet another advantage to provide a hand-held laser surgical device for use with a surgical laser tip apparatus with alignment assembly, such as our surgical laser tip apparatus with alignment assembly disclosed and claimed in pending U.S. Non-provisional patent application Ser. No. 12/257,665, filed on Oct. 244, 2008 [“'665 Application”] which is incorporated herein by reference for all purposes.

For a more complete understanding of the above and other features, advantages, and objects of the invention, reference should be made to the following detailed description of a preferred embodiment, and to the accompanying drawings.

### BRIEF DESCRIPTION OF DRAWINGS

FIG. 1 is a side elevation view of an embodiment of a hand-held, portable laser surgical apparatus 10 depicting a handle portion 12, including a detachable power supply housing 14, interactive control and feedback assembly 18, disposable sterile tip assembly 20, optical fiber 22, and assembly 26 for aligning the optical fiber with the handle portion 12.

FIG. 2 is a sectional view of FIG. 5 taken at A′-A.′

FIG. 3 is an exploded perspective view of FIG. 1.

FIG. 4 is an exploded view of the laser assembly 24 for releasably attaching the disposable sterile tip assembly 20 to the handle portion 12 and aligning the optical fiber 22 with at least one laser source 16.

FIG. 5 is a perspective view of FIG. 1 depicting the power supply housing 14, the disposable sterile tip 20, and the handle portion 12.

FIG. 6 is a perspective view of the base charging unit 30 housing a hand-held, portable laser surgical apparatus 10 and depicting a second power supply 15 being recharged.

FIG. 7 is a perspective view of perspective view of FIG. 1 receiving the radio waves from foot control transmitter 28.

### DETAILED DESCRIPTION OF THE INVENTION

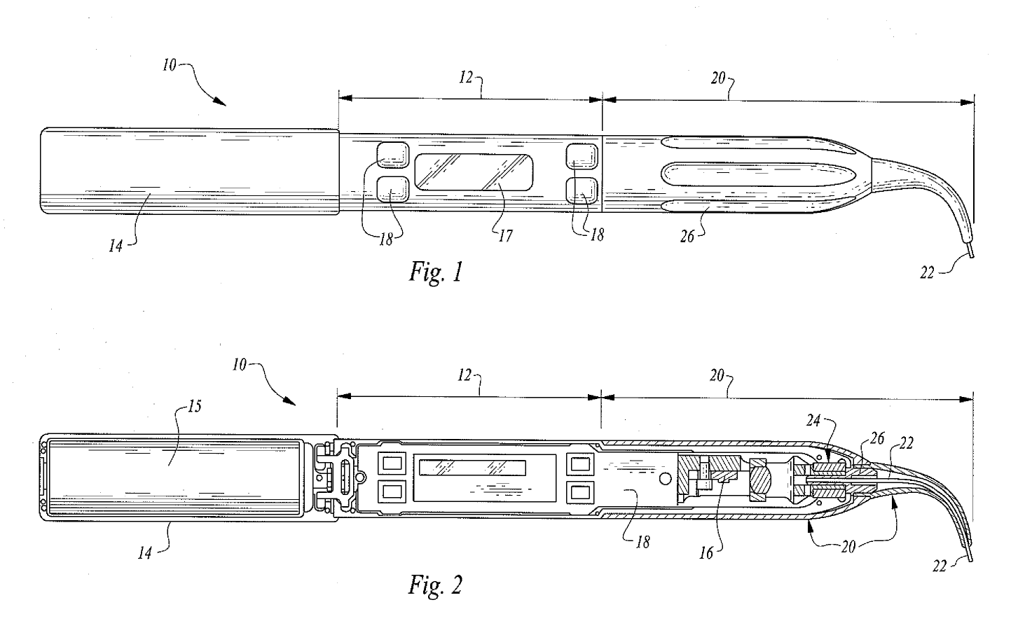

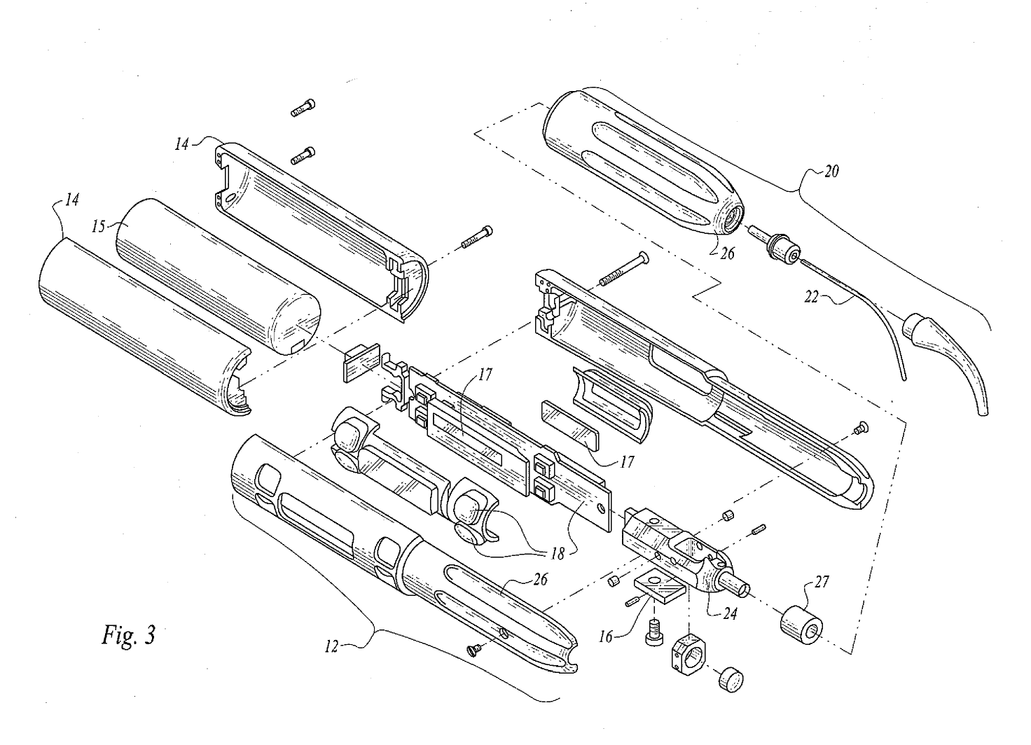

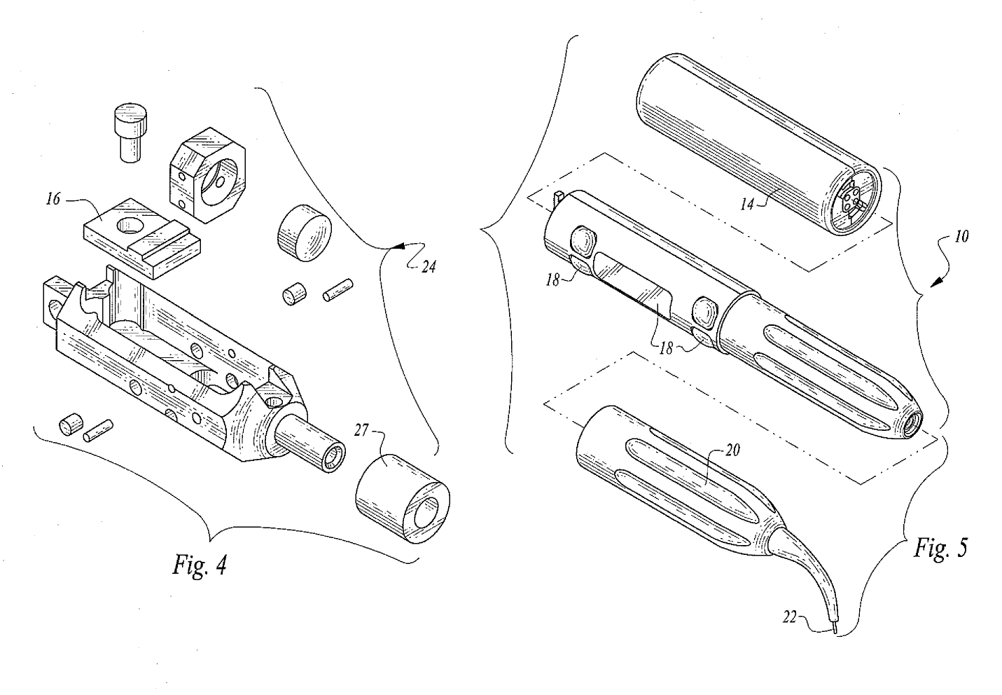

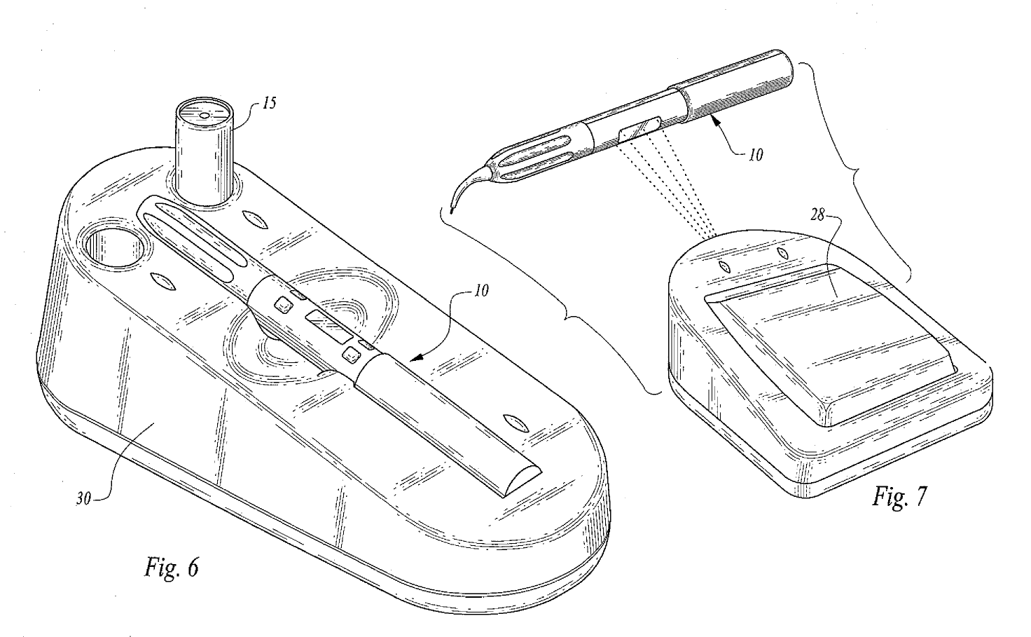

With reference to drawing FIGS. 1-7, a hand-held, portable laser surgical apparatus 10, foot pedal 28, and base charger 30 are presented. The reference numeral 10 designates generally typical apparatus for a hand-held, portable laser surgical device.

Our disposable surgical tip apparatus disclosed and claimed in the '336 Application is incorporated herein by reference for all purposes.

Our dual diode converging module disclosed and claimed in the '383 Application is incorporated herein by reference for all purposes.

Our dual surgical laser tip apparatus with alignment assembly disclosed and claimed in the '665 Application is incorporated herein by reference for all purposes.

The hand-held, portable laser surgical apparatus 10, comprises in combination: a handle portion 12; an integral, detachable and rechargeable power supply 15 within the handle portion power housing 14; at least one self contained laser source within the handle portion 16; interactive control and feedback assembly 18 of the apparatus function; disposable sterile tip assembly 20; optical fiber for transmitting a generated laser beam 22; assembly 24 for releasably attaching the disposable sterile tip assembly 20 to the handle portion 12; assembly 26 for aligning optical fiber with at least one laser source 16; assembly 28 for wireless foot peddle control of the apparatus 10 on/off function; and assembly 30 for sterilization, recharging and storage of the apparatus when the apparatus is not in use. The preferred embodiment includes 400 micron fiber delivery system for the optical fiber 22.

It is understood by persons having ordinary skill in the art that the power supply 15 communicates with circuitry (not shown) for the laser source(s) 16 and interactive control and feedback assembly.

For an embodiment of the apparatus 10, at least one self contained laser source comprises at least one laser diode.

The self contained laser source for an embodiment of the apparatus 10 includes at least one laser diode that generates an optimal output aiming beam range between approximately 800 nm infrared beam and 3100 nm infrared beam with an output power range between 1 watt and 15 watts.

For an alternate embodiment of the apparatus 10, the self contained laser source includes at least a second laser diode that generates an optimal output aiming beam within the visible spectrum of a laser diode with an output power range between 1 mW and 25 mW.

For an embodiment of the apparatus 10, the assembly for interactive control and feedback 18 of the apparatus function comprises at least one light emitting diode (“LED”) display 17. The preferred embodiment of the apparatus 10 includes feedback assembly 18 LED display 17 that adapts the display to adjust to the hand holding the apparatus 10 providing the user with a readable output on the screen.

For an embodiment of the apparatus 10, the assembly for interactive control and feedback 18 of the apparatus function further comprises at least one operational button.

The preferred embodiment of the apparatus 10 includes a rechargeable Lithium ion power supply 15.

The preferred embodiment of the apparatus 10 includes a base charger 30 having approximate dimensions of 2.7 inches in height, 2.9 inches in width, and 8 inches in length. The base charger 30 uses 100-240 VAC at 50 to 60 Hz and maximum amps of 0.8 to recharge the Lithium ion power supply 15 and bathe the apparatus 10 with ultra violet (UV) light for additional cleanliness and sterilization.

An embodiment of the apparatus 10 includes assembly for wireless foot peddle control 28 of the apparatus on/off function that comprises an assembly for radio wave reception and transmission. The preferred embodiment of assembly for wireless foot peddle control 28 includes a wireless frequency of 2.4 GHz, and an internal power supply of AA batteries.

For an embodiment of the apparatus 10, the assembly for radio wave reception and transmission further comprises an assembly for Bluetooth™ communication to receive the wireless foot peddle control 28 wireless frequency signal.

For an embodiment of the apparatus 10, the disposable sterile tip assembly 20 houses the optical fiber 22 for transmitting generated laser beam, and the disposable sterile tip assembly 20 comprises prophylactic packaging.

For an embodiment of the apparatus 10, the assembly for releasably attaching the means for disposable sterile tip assembly 20 to the handle portion comprises a magnetic assembly.

For an embodiment of the apparatus 10, the handle portion comprises a distal end, the assembly for disposable sterile tip assembly 20 comprises a first end sleeve, and the assembly for aligning optical fiber with at least one laser source comprises at least two slits spaced equally apart on the distal end of the handle portion and a tip first end sleeve having internal longitudinal ribs adapted to fit the handle portion slits.

For an embodiment of the apparatus 10, the assembly 27 for releasably attaching the disposable sterile tip assembly 20 to the handle portion 12 comprises at least one member of the group consisting of assembly for magnetic coupling, assembly for mechanical coupling, assembly for electromechanical coupling, and assembly for electromagnetic coupling.

The preferred embodiment of the handle portion 12 and connected power supply housing 14 of the apparatus 10 weighs 1.9 ounces, and measures approximately 6.3 inches in length and approximately 0.65 inches in diameter.

It should be understood, of course, that the specific forms of the invention illustrated herein and described are intended to be representative only, as certain changes may be made therein without departing from the clear teachings of the disclosure. Accordingly, reference should be made to the following appended claims in determining the full scope of the invention.

1-6. (canceled)

7. A hand-held, portable laser surgical apparatus, the apparatus:
a handle having a generally cylindrical shape with a first end and a second end;
a power supply housing removably coupled to the exterior of said first end of said handle and extending distally from said first end of said handle;
at least one rechargeable power supply located within said power supply housing;
at least one self contained laser source located within said handle, wherein said at least one self contained laser source further comprising at least one output diode which outputs a laser beam with an output power range between 1 and 15 watts;
at least one LED display located on said handle, wherein the orientation of the output of said LED display adjusts depending whether said handle is held by a user's left hand or right hand; and
said second end of said handle further comprising a mechanism for releasably coupling a disposable sterile tip to said second end of said handle.

8. The hand-held, portable laser surgical apparatus of claim 7, wherein said at least one output diode outputs an optimal output laser beam between approximately 800 nm beam and 3100 nm beam.

9. The hand-held, portable laser surgical apparatus of claim 7, wherein said self-contained laser source further comprises at least a one aiming diode for outputting an aiming beam within the visible spectrum.

10. The apparatus of claim 9, wherein said aiming beam is has an output power range between 1 mW and 25 mW.

11. The hand-held, portable laser surgical apparatus of claim 7, wherein said handle further comprises at least at least one button.

12. The hand-held, portable laser surgical apparatus of claim 7, wherein said handle further comprises a means for wireless communication with a foot pedal for control of the apparatus.

13. The hand-held, portable laser surgical apparatus of claim 12, wherein said means for wireless communication is selected from the group consisting of radio wave, infrared or Bluetooth.

14. The hand-held, portable laser surgical apparatus of claim 7, wherein said disposable sterile tip further comprises a generally cylindrical shape about a longitudinal axis, a first end sleeve and a second end which tapers to a discharge tip containing an optical fiber.

15. The hand-held, portable laser surgical apparatus of claim 14, wherein said first end sleeve further comprises an alignment assembly for aligning said optical fiber contained within said discharge tip with said at least one self contained laser source.

16. The hand-held, portable laser surgical apparatus of claim 15, where said alignment assembly further comprises at least two internal longitudinal ribs.

17. The hand-held, portable laser surgical apparatus of claim 16, wherein said second end of said handle comprises at least two slits adapted to fit said at least two internal longitudinal ribs on said first end sleeve of said disposable sterile tip.

18. The hand-held, portable laser surgical apparatus of claim 7, wherein said mechanism for releasably coupling said disposable sterile tip to said second end of said handle comprises at least one member of the group consisting of the following mechanisms: magnetic, mechanical, electro-mechanical, or electromagnetic.

19. The hand-held, portable laser surgical apparatus of claim 7, wherein said disposable sterile tip is packaged in a prophylactic package

20. The hand-held, portable laser surgical apparatus of claim 7, further comprising a base charger for storing and charging said rechargeable power supply.

21. The hand-held, portable laser surgical apparatus of claim 20, wherein said base charger further comprises an assembly for storage of said handle.

22. The hand-held, portable laser surgical apparatus of claim 20 wherein said base charger further comprises a UV light.

23. The hand-held, portable laser surgical apparatus of claim 7 wherein said handle coupled with said power supply housing have a combined length of about 6.3 inches.

24. The hand-held, portable laser surgical apparatus of claim 7 wherein said handle coupled with said power supply housing have a combined weight of about 1.9 ounces.

25. The hand-held, portable laser surgical apparatus of claim 7 wherein said handle has a diameter of about 0.65 inches.

26. The hand-held, portable laser surgical apparatus of claim 14, wherein said discharge tip is at an acute angle relative to said longitudinal axis.

27. The hand-held, portable laser surgical apparatus of claim 14, wherein said discharge tip is at about a 90° angle relative to said longitudinal axis.
